(19) United States
(12) Patent Application Publication
(10) Pub. No.: US 2025/0303137 A1
(43) Pub. Date: Oct. 2, 2025

(54) METHODS AND SYSTEMS FOR CORRECTING SENSOR DRIFT FOR AN INTRAVASCULAR BLOOD PUMP

(71) Applicant: Abiomed, Inc., Danvers, MA (US)

(72) Inventors: Matthew Crema, Danvers, MA (US); Ahmad El Katerji, Danvers, MA (US); Mohammed Alwatban, Danvers, MA (US); Yuting Zhang, Danvers, MA (US); Daniel Podlisny, Danvers, MA (US); Atahar Mostafa, Danvers, MA (US)

(21) Appl. No.: 19/091,208

(22) Filed: Mar. 26, 2025

Related U.S. Application Data
(60) Provisional application No. 63/571,809, filed on Mar. 29, 2024.

(51) Int. Cl. A61M 60/531; A61M 60/585

(52) U.S. Cl. CPC A61M 60/531; A61M 60/585; A61M 2205/702

(57) ABSTRACT

Methods and apparatus for correcting sensor drift associated with a pressure sensor of a heart pump are provided. The method includes recording, at a first time, a first reference signature for a pressure signal received from the pressure sensor, receiving, at a second time, an indication to correct sensor drift associated with the pressure sensor, wherein the second time is after the first time, receiving a real-time pressure signal from the pressure sensor, adjusting, by a controller associated with the heart pump, the real-time pressure signal based on the first reference signature in response to receiving the indication to correct sensor drift, and displaying the adjusted real-time pressure signal.

### CROSS REFERENCE TO RELATED APPLICATIONS

This application claims the benefit under 35 U.S.C. § 119 (c) to U.S. Provisional Patent Application No. 63/571,809, filed Mar. 29, 2024, and titled, “METHODS AND SYSTEMS FOR CORRECTING SENSOR DRIFT FOR AN INTRAVASCULAR BLOOD PUMP,” the entire contents of which is incorporated by reference herein.

### FIELD OF THE INVENTION

This disclosure relates to techniques for correcting sensor drift for an intravascular blood pump.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

An intravascular blood pump may include one or more pressure sensors configured to sense pressure values inside a patient's heart during placement and/or operation of the blood pump. Sensitivity of the pressure sensor(s) to changes in temperature and/or other factors may result in the pressure signals sensed by such pressure sensors to experience sensor drift (e.g., a DC offset) over time. For example, a sensor mounting process used to mount the pressure sensor on the blood pump may result in varying thicknesses of a silicone membrane across pumps, which may result in sensors for different pumps having different sensor drift characteristics. Sensor drift may impact the accuracy of the operational data and/or alarms presented on a graphical user interface of the controller associated with the blood pump. Described herein are systems and methods for detecting sensor drift associated with a pressure signal sensed by a pressure sensor of an intravascular blood pump based, at least in part, on an analysis of the pressure signal. Although the techniques described herein are used to detect sensor drift in a differential pressure signal of a pressure sensor for a blood pump inserted across the pulmonary valve in the right side of the heart, it should be appreciated that at least some of the techniques may also be used to detect sensor drift in a signal of a different type of sensor associated with an intravascular blood pump (e.g., a pressure sensor of a blood pump inserted across the aortic valve in the left side of the heart). Also described herein are systems and methods for correcting for sensor drift of a pressure signal sensed by a pressure sensor of an intravascular blood pump.

In one aspect, a method of correcting sensor drift associated with a pressure sensor of a heart pump. The method includes recording, at a first time, a first reference signature for a pressure signal received from the pressure sensor, receiving, at a second time, an indication to correct sensor drift associated with the pressure sensor, wherein the second time is after the first time, receiving a real-time pressure signal from the pressure sensor, adjusting, by a controller associated with the heart pump, the real-time pressure signal based on the first reference signature in response to receiving the indication to correct sensor drift, and displaying the adjusted real-time pressure signal.

In another aspect, the pressure sensor comprises a differential pressure sensor. In another aspect, the heart pump is inserted across a pulmonary valve of a heart of a patient, and the pressure signal is a differential pressure signal across the pulmonary valve. In another aspect, recording the first reference signature comprises calculating a sum of pressure signal values within a time window of the pressure signal, the time window including the first time. In another aspect, recording the first reference signature comprises determining a mean placement signal within a time window of the pressure signal, the time window including the first time. In another aspect, receiving the indication to correct sensor drift in the pressure signal comprises receiving user input via a user interface associated with the heart pump. In another aspect, the method further includes detecting, based on an analysis of the pressure signal by the controller, the sensor drift, wherein receiving the indication to correct sensor drift in the pressure signal comprises receiving the indication from the controller in response to detecting the sensor drift. In another aspect, automatically adjusting the real-time pressure signal based on the first reference signature in response to receiving the indication to correct sensor drift comprises re-centering the real-time pressure signal based on the first reference signature. In another aspect, re-centering the real-time pressure signal based on the first reference signature comprises subtracting or adding an offset value based on the first reference signature to the real-time pressure signal.

In another aspect, the method further includes determining whether to perform automatic drift calibration, and outputting an alert on a user interface associated with the heart pump when it is determined not to perform automatic drift calibration. In another aspect, adjusting the real-time pressure signal based on the first reference signature is performed without adjusting a speed of the heart pump. In another aspect, the method further includes recording, at the second time, a second reference signature for the pressure signal, receiving, at a third time, an indication to correct sensor drift associated with the pressure sensor, wherein the third time is after the second time, and adjusting, by the controller associated with the heart pump, the real-time pressure signal based on the second reference signature in response to receiving, at the third time, the indication to correct sensor drift. In another aspect, the method further includes detecting, at a third time, a change in a speed of the heart pump, wherein the third time is after the first time, recording, at the third time, a second reference signature for the pressure signal, receiving, at a fourth time, an indication to correct sensor drift associated with the pressure sensor, wherein the fourth time is after the third time, and adjusting, by the controller associated with the heart pump, the real-time pressure signal based on the second reference signature in response to receiving, at the fourth time, the indication to correct sensor drift.

In another aspect, the method further includes determining at a third time, between the first time and the second time, a signature within a time window of the pressure signal, updating a minimum signature value or a maximum signature value based, at least in part, on the signature within the time window, and determining whether a range of the signature is greater than a threshold value, wherein the range is determined as a difference between the minimum signature value and the maximum signature value, wherein an indication to correct sensor drift associated with the pressure sensor comprises receiving the indication in response to determining that the range of the signature is greater than the threshold value. In another aspect, determining a signature within a time window of the pressure signal comprises determining a sum of pressure signal values within the time window of the pressure signal. In another aspect, determining a signature within a time window of the pressure signal comprises determining a mean value of pressure signal values within the time window of the pressure signal. In another aspect, the method further includes initializing, at the first time, the minimum signature value and the maximum signature value to a same value, and updating the minimum signature value and/or the maximum signature value based, at least in part, on the signature comprises updating the minimum signature value to the mean value when the mean value is less than the minimum signature value or updating the maximum signature value to the mean value when the mean value is greater than the maximum signature value. In another aspect, a length of the time window is one minute.

In one aspect, a heart pump system is provided. The heart pump system includes a heart pump including a pressure sensor configured to sense a pressure within a portion of a heart of a patient and a controller. The controller is configured to record, at a first time, a first reference signature for a pressure signal received from the pressure sensor, receive, at a second time, an indication to correct sensor drift associated with the pressure sensor, wherein the second time is after the first time, receive a real-time pressure signal from the pressure sensor, adjust, by a controller associated with the heart pump, the real-time pressure signal based on the first reference signature in response to receiving the indication to correct sensor drift, and display the adjusted real-time pressure signal.

In another aspect, the pressure sensor comprises a differential pressure sensor. In another aspect, the heart pump is configured to be inserted across a pulmonary valve of a heart of a patient, and the pressure signal is a differential pressure signal across the pulmonary valve. In another aspect, recording the first reference signature comprises calculating a sum of pressure signal values within a time window of the pressure signal, the time window including the first time. In another aspect, recording the first reference signature comprises determining a mean placement signal within a time window of the pressure signal, the time window including the first time. In another aspect, receiving the indication to correct sensor drift in the pressure signal comprises receiving user input via a user interface associated with the heart pump. In another aspect, the controller is further configured to detect the sensor drift based on an analysis of the pressure signal, and receiving the indication to correct sensor drift in the pressure signal comprises receiving the indication from the controller in response to detecting the sensor drift. In another aspect, automatically adjusting the real-time pressure signal based on the first reference signature in response to receiving the indication to correct sensor drift comprises re-centering the real-time pressure signal based on the first reference signature. In another aspect, re-centering the real-time pressure signal based on the first reference signature comprises subtracting or adding an offset value based on the first reference signature to the real-time pressure signal.

In another aspect, the controller is further configured to determine whether to perform automatic drift calibration and output an alert on a user interface associated with the heart pump when it is determined not to perform automatic drift calibration. In another aspect, adjusting the real-time pressure signal based on the first reference signature is performed without adjusting a speed of the heart pump. In another aspect, the controller is further configured to record, at the second time, a second reference signature for the pressure signal, receive, at a third time, an indication to correct sensor drift associated with the pressure sensor, wherein the third time is after the second time, and adjust, by the controller associated with the heart pump, the real-time pressure signal based on the second reference signature in response to receiving, at the third time, the indication to correct sensor drift.

In another aspect, the controller is further configured to detect, at a third time, a change in a speed of the heart pump, wherein the third time is after the first time, record, at the third time, a second reference signature for the pressure signal, receive, at a fourth time, an indication to correct sensor drift associated with the pressure sensor, wherein the fourth time is after the third time, and adjust the real-time pressure signal based on the second reference signature in response to receiving, at the fourth time, the indication to correct sensor drift. In another aspect, the controller is further configured to determine at a third time, between the first time and the second time, a signature within a time window of the pressure signal, update a minimum signature value or a maximum signature value based, at least in part, on the signature within the time window, and determine whether a range of the signature is greater than a threshold value, wherein the range is determined as a difference between the minimum signature value and the maximum signature value, wherein an indication to correct sensor drift associated with the pressure sensor comprises receiving the indication in response to determining that the range of the signature is greater than the threshold value. In another aspect, determining a signature within a time window of the pressure signal comprises determining a sum of pressure signal values within the time window of the pressure signal. In another aspect, determining a signature within a time window of the pressure signal comprises determining a mean value of pressure signal values within the time window of the pressure signal. In another aspect, the controller is further configured to initialize, at the first time, the minimum signature value and the maximum signature value to a same value, wherein updating the minimum signature value and/or the maximum signature value based, at least in part, on the signature comprises updating the minimum signature value to the mean value when the mean value is less than the minimum signature value or updating the maximum signature value to the mean value when the mean value is greater than the maximum signature value. In another aspect, a length of the time window is one minute.

In one aspect, a controller for a heart pump system is provided. The controller includes at least one hardware processor. The at least one hardware processor is configured to record, at a first time, a first reference signature for a pressure signal received from a pressure sensor associated with the heart pump system, receive, at a second time, an indication to correct sensor drift associated with the pressure sensor, wherein the second time is after the first time, receive a real-time pressure signal from the pressure sensor, adjust the real-time pressure signal based on the first reference signature in response to receiving the indication to correct sensor drift, and display the adjusted real-time pressure signal.

In another aspect, the pressure sensor comprises a differential pressure sensor. In another aspect, the heart pump system includes a heart pump configured to be inserted across a pulmonary valve of a heart of a patient, and the pressure signal is a differential pressure signal across the pulmonary valve. In another aspect, recording the first reference signature comprises calculating a sum of pressure signal values within a time window of the pressure signal, the time window including the first time. In another aspect, recording the first reference signature comprises determining a mean placement signal within a time window of the pressure signal, the time window including the first time. In another aspect, receiving the indication to correct sensor drift in the pressure signal comprises receiving user input via a user interface associated with the heart pump system. In another aspect, the at least one hardware processor is further configured to detect the sensor drift based on an analysis of the pressure signal, and receiving the indication to correct sensor drift in the pressure signal comprises receiving the indication from the controller in response to detecting the sensor drift. In another aspect, automatically adjusting the real-time pressure signal based on the first reference signature in response to receiving the indication to correct sensor drift comprises re-centering the real-time pressure signal based on the first reference signature. In another aspect, re-centering the real-time pressure signal based on the first reference signature comprises subtracting or adding an offset value based on the first reference signature to the real-time pressure signal.

In another aspect, the at least one hardware processor is further configured to determine whether to perform automatic drift calibration and output an alert on a user interface associated with the heart pump system when it is determined not to perform automatic drift calibration. In another aspect, adjusting the real-time pressure signal based on the first reference signature is performed without adjusting a speed of a heart pump of the heart pump system. In another aspect, the at least one hardware processor is further configured to record, at the second time, a second reference signature for the pressure signal, receive, at a third time, an indication to correct sensor drift associated with the pressure sensor, wherein the third time is after the second time, and adjust the real-time pressure signal based on the second reference signature in response to receiving, at the third time, the indication to correct sensor drift.

In another aspect, the at least one hardware processor is further configured to detect, at a third time, a change in a speed of a heart pump of the heart pump system, wherein the third time is after the first time, record, at the third time, a second reference signature for the pressure signal, receive, at a fourth time, an indication to correct sensor drift associated with the pressure sensor, wherein the fourth time is after the third time, and adjust the real-time pressure signal based on the second reference signature in response to receiving, at the fourth time, the indication to correct sensor drift.

In another aspect, the at least one hardware processor is further configured to determine at a third time, between the first time and the second time, a signature within a time window of the pressure signal, update a minimum signature value or a maximum signature value based, at least in part, on the signature within the time window, and determine whether a range of the signature is greater than a threshold value, wherein the range is determined as a difference between the minimum signature value and the maximum signature value, wherein an indication to correct sensor drift associated with the pressure sensor comprises receiving the indication in response to determining that the range of the signature is greater than the threshold value. In another aspect, determining a signature within a time window of the pressure signal comprises determining a sum of pressure signal values within the time window of the pressure signal. In another aspect, determining a signature within a time window of the pressure signal comprises determining a mean value of pressure signal values within the time window of the pressure signal. In another aspect, the at least one hardware processor is further configured to initialize, at the first time, the minimum signature value and the maximum signature value to a same value, and updating the minimum signature value and/or the maximum signature value based, at least in part, on the signature comprises updating the minimum signature value to the mean value when the mean value is less than the minimum signature value or updating the maximum signature value to the mean value when the mean value is greater than the maximum signature value. In another aspect, a length of the time window is one minute.

### BRIEF DESCRIPTION OF DRAWINGS

FIG. 1A shows an illustrative cardiac support device that may be used with some embodiments.

FIG. 1B shows an illustrative cardiac support system that includes the cardiac support device of FIG. 1A.

FIG. 2A shows a plot of a motor speed signal for a cardiac support device, in accordance with some embodiments.

FIG. 2B shows a plot of a differential pressure signal sensed by a pressure sensor of a cardiac support device, in accordance with some embodiments.

FIG. 2C shows a plot of a motor current signal associated with a cardiac support device, in accordance with some embodiments.

FIG. 3 is a flowchart of a process for detecting sensor drift associated with a sensor of a cardiac support device, in accordance with some embodiments.

FIG. 4 is a flowchart of a process for detecting sensor drift using a sample summation technique, in accordance with some embodiments.

FIG. 5 is a flowchart of a process for detecting sensor drift using a mean placement signal technique, in accordance with some embodiments.

FIG. 6A is a plot illustrating an example of using the mean placement signal technique of FIG. 5 to detect sensor drift, in accordance with some embodiments.

FIG. 6B is a plot illustrating the mean motor current associated with a cardiac support device during a time period associated with the plot shown in FIG. 6A.

FIG. 7 is a flowchart of a process for correcting sensor drift associated with a sensor of a cardiac support device, in accordance with some embodiments.

### DETAILED DESCRIPTION

Physicians and other healthcare providers may rely on indications of the operational status and/or patient physiological parameters displayed by a controller of a cardiac support device (e.g., an intravascular blood pump) to ensure that the device is properly placed in the patient's heart and/or to determine whether settings of the device (e.g., pump speed) should be adjusted as the device provides support to the patient. The cardiac support device may include one or more sensors (e.g., pressure sensors) configured to sense a pressure within one or more chambers of the patient's heart, and the sensed pressure signal may be used to determine one or more of the indications of operational status and/or patient physiological parameters displayed by the controller. For instance, the controller may be configured to display an indication of a patient's central venous pressure (CVP) and pulmonary artery pressure (PAP) determined based, at least in part, on a differential pressure signal sensed by a pressure sensor of a cardiac support device inserted in the right side of a heart of a patient. The differential pressure signal may reflect a pressure difference across the pulmonary valve of the patient's heart. The displayed CVP and PAP values and/or waveforms may be updated by a controller in real-time (or near real-time) as differential pressure sensor measurements are continuously sensed by the pressure sensor during operation of the cardiac support device.

Over time the differential pressure signal sensed by the differential pressure sensor may drift from a reference (e.g., baseline) value, which may result in inaccurate estimates of various physiological parameters such as CVP and PAP. Some conventional techniques for detecting sensor drift for a pressure sensor of a cardiac support device may require the user to notice erroneous values (e.g., large negative values for CVP and/or PAP) being displayed on the controller. The inventors have recognized and appreciated that because sensor drift tends to manifest in the sensed pressure signal (and physiological measurements based on the sensed pressure signal) over a relatively long time span, manual detection of sensor drift is challenging, particularly when the user is not focused on detecting sensor drift. To this end, some embodiments of the present disclosure relate to novel data-driven techniques for detecting sensor drift based on an analysis of the sensed pressure signal.

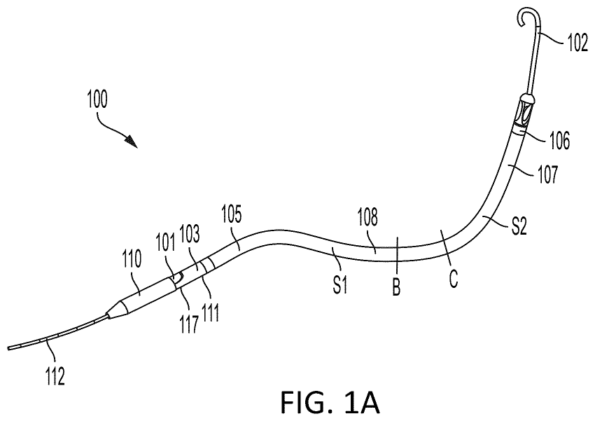

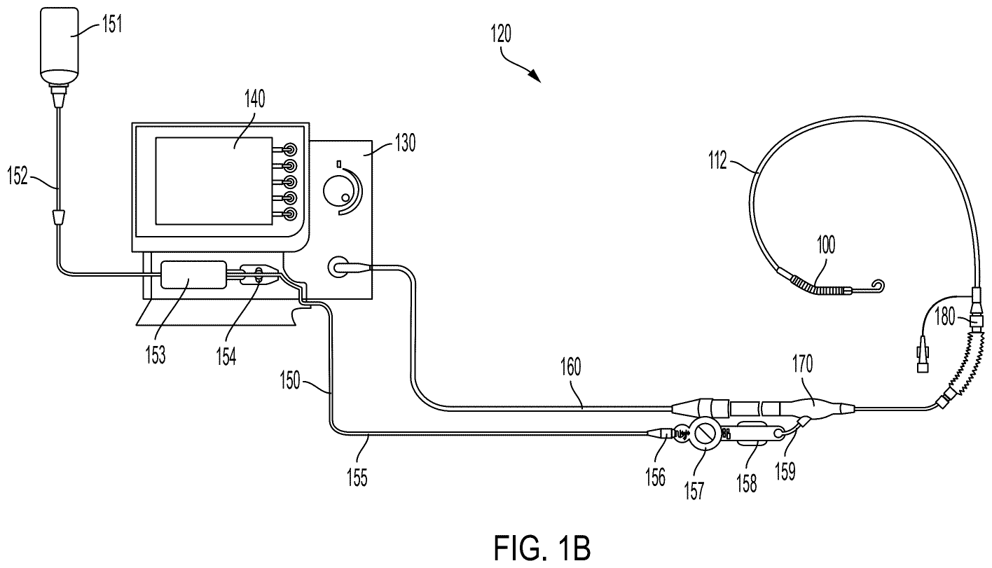

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the wall 111 of the pump housing 103. The pump housing 103 may be composed of a metal in accordance with some implementations. The extension 102, also referred to as a “pigtail,” may be connected to the distal end 107 of the cannula 108 and may assist with stabilizing and/or positioning the blood pump assembly 100 into the correct position in the heart. The pigtail may be configurable from a straight to a partially curved configuration. The pigtail may be composed, at least in part of a flexible material, and may have dual stiffness. It should be appreciated that some embodiments of the pump assembly may not include a pigtail.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or) 170°. The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieved the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to receive measurements from one or more pressure sensors (not shown) included as a portion of blood pump assembly 100 and purge disc 154. Controller 130 may also be configured to control operation of the motor (not shown) of the blood pump assembly 100 and purge cassette 153. In some embodiments, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

As described herein, a heart pump (e.g., blood pump assembly 100) may include a pressure sensor (e.g., a differential pressure sensor) configured to detect a pressure difference between an inlet and an outlet of the heart pump when the pump is placed across a valve in a patient's heart. For instance, when a right heart cardiac support device is positioned properly, the inlet of the pump may be positioned within right atrium of the patient's heart, and the outlet of the heart pump may be positioned within the pulmonary artery of a patient's heart, with the pump spanning the pulmonary valve. The pressure signal sensed by the differential pressure sensor may be used, at least in part, to determine correct positioning of the heart pump within the patient's heart and/or to determine various pressure metrics (e.g., CVP, PAP) that may be displayed to a user during operation of the pump.

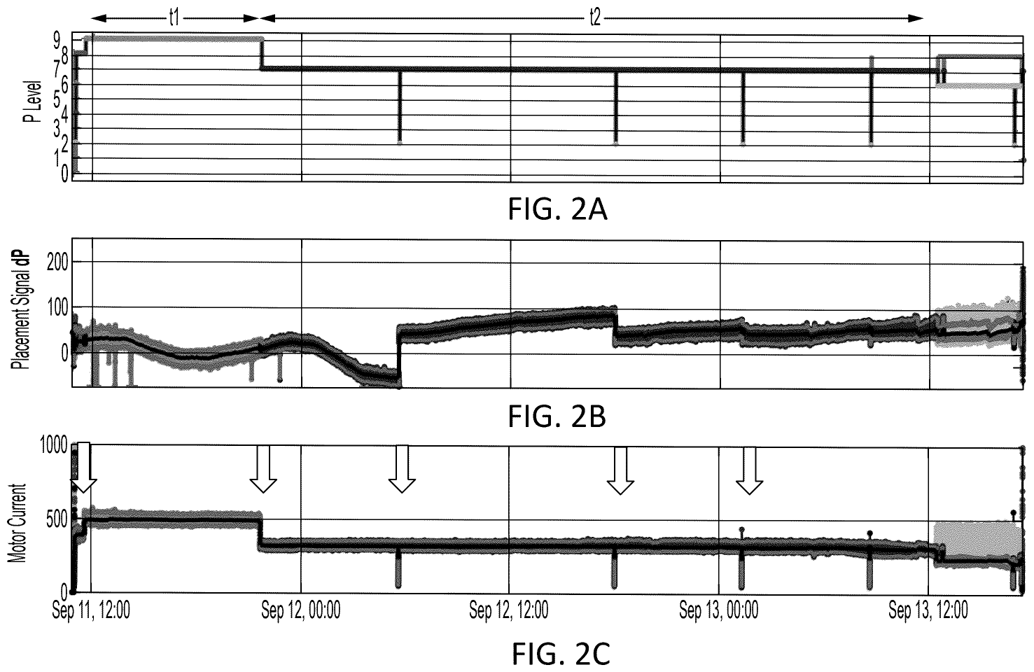

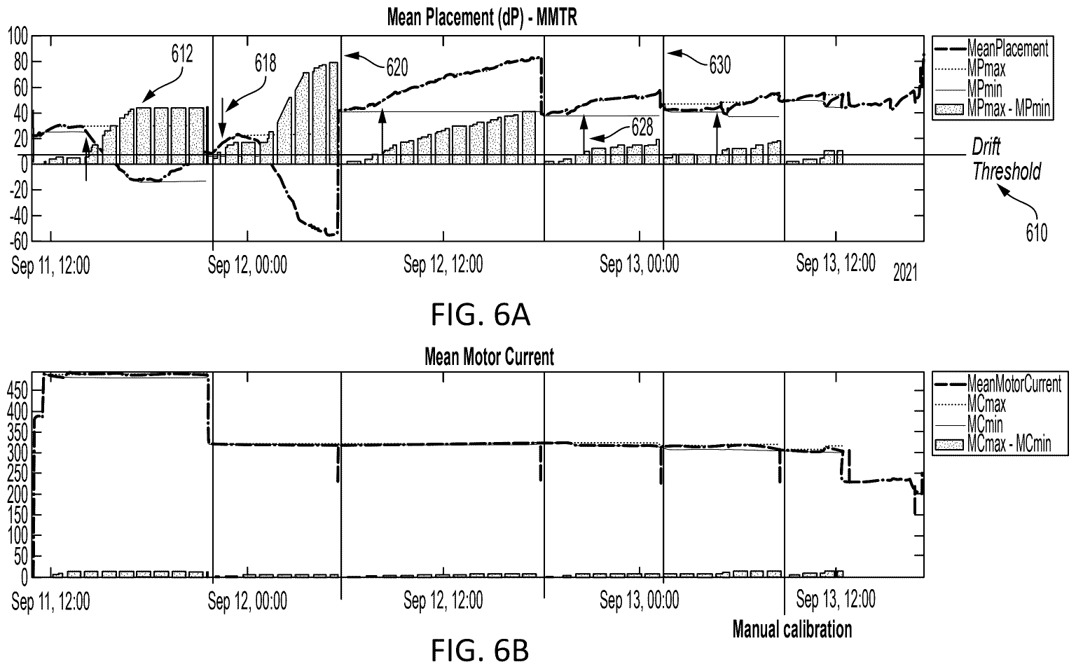

As described in connection with FIGS. 1A and 1B, a cardiac support system (e.g., cardiac support system 120) may include a controller (e.g., controller 130) configured to control operation of a motor of a heart pump to spin an impeller of the heart pump at a particular speed (referred to herein as P-levels, with P0 being the slowest speed and P9 being the fastest speed), thereby affecting the rate at which blood is pumped from the inlet to the outlet of the heart pump. FIG. 2A shows a plot of P-level as a function of time for an example operation of a heart pump system. As shown in FIG. 2A, the pump was operated at P-level of P9 for a time period t1, then was operated at a P-level of P7 for a time period t2. FIG. 2B shows a plot of a differential pressure (dP) signal sensed by a differential pressure sensor of the heart pump during the time periods t1 and t2 shown in FIG. 2A. FIG. 2C shows a plot of a motor current signal during the same time period t1 and t2. As can be observed in FIG. 2B, the differential pressure signal (dP) drifts during each of the time periods t1 and t2, whereas as can be observed in FIG. 2C, the motor current remains relatively constant during each of the time periods t1 and t2. The arrows in FIG. 2C indicate time points at which a calibration of a reference value (e.g., a DC reference value) associated with the differential pressure signal shown in FIG. 2B was performed. As shown, the reference value may be recalibrated each time the P-level is changed. The reference value may also be recalibrated at other times when the user determines that such recalibration is necessary due to sensor drift. As discussed herein, in conventional systems, sensor drift is not automatically detected, and as such, determining when to recalibrate the reference value for the differential pressure signal is left up to the user's discretion when they notice an issue with one or more displayed signals. As discussed in connection with FIGS. 6A and 6B, the automatic sensor drift detection techniques described herein may be capable of detecting sensor drift considerably faster than relying on conventional user identification techniques.

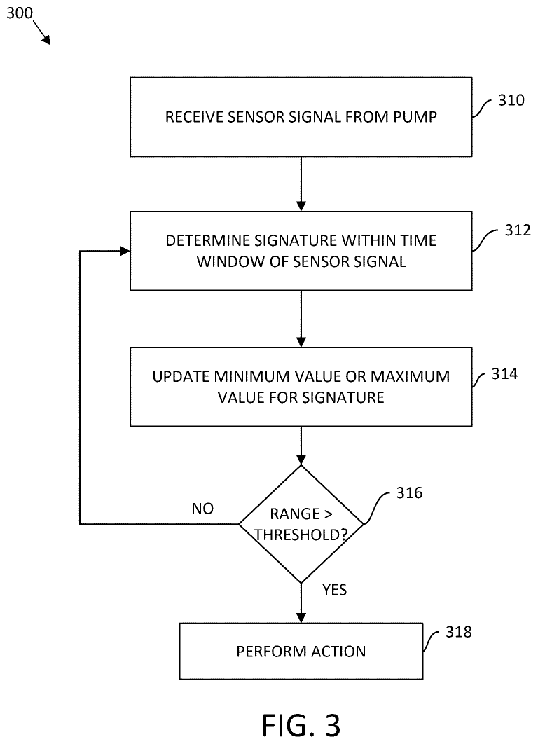

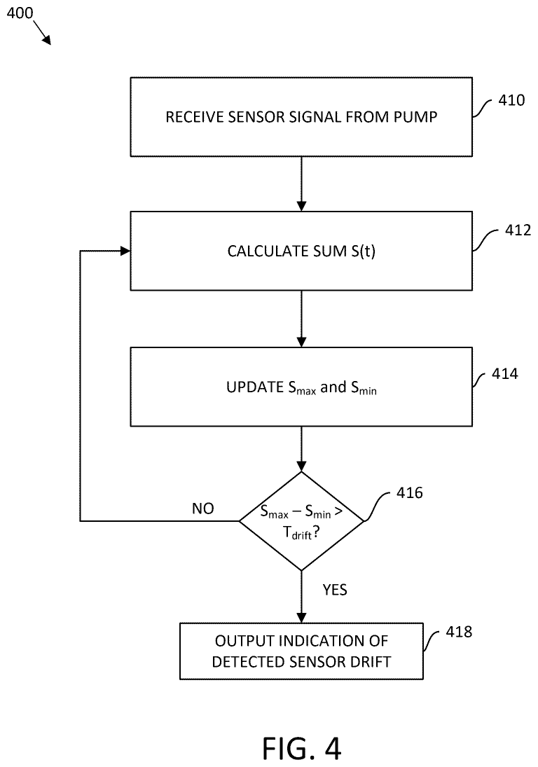

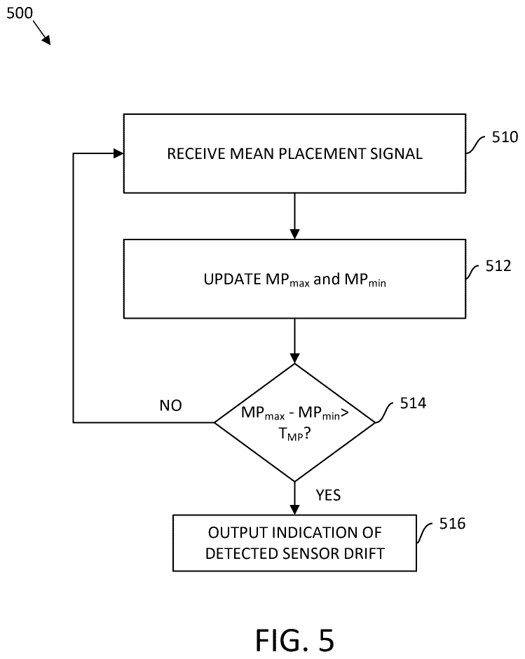

FIG. 3 illustrates a process 300 for detecting sensor drift in a pressure sensor signal, in accordance with some embodiments. Process 300 may begin in act 310, where a sensor signal is received from a heart pump. As described herein, the heart pump may have one or more pressure sensors (e.g., a differential pressure sensor) arranged thereon. During placement and/or operation of the heart pump, the one or more pressure sensors may be configured to output a pressure signal that is sent to a controller (e.g., controller 130 shown in FIG. 1B) associated with the heart pump. In some embodiments, the controller may be configured to process the received signal to detect sensor drift (e.g., by performing process 300 shown in FIG. 3). After receiving the sensor signal, process 300 may proceed to act 312, where a signature (e.g., a DC signature) may be determined within a time window of the received sensor signal. As shown in the example of FIG. 2B, sensor drift tends to occur over a relatively long time scale (e.g., hours, days, etc.). The inventors have recognized that signatures of drift determined over a shorter time window (e.g., seconds, minutes, etc.) may be used to approximate the sensor drift tendency over the longer time scale. For example, in some embodiments, a random-phase approximation signature, which may use semi-local density function computations to approximate a global function representing sensor drift, may be determined in act 312. In some embodiments, the signature may be calculated as a statistical value (e.g., sum, mean, etc.) over a time window that characterizes the sensor values within the window. Process 400 shown in FIG. 4 and process 500 shown in FIG. 5 provide further details of some examples for determining a signature, in accordance with some embodiments of the present disclosure.

Process 300 then proceeds to act 314, where a minimum value or maximum value for the signature may be updated based, at least in part, on the signature determined in act 312. Some embodiments track a range of values for the signature over time to detect drift in the sensor signal. For instance, a minimum signature value and a maximum signature value may be stored and updated each time a new signature value is determined in act 312 if the newly-determined signature value is less than the stored minimum value or is greater than the stored maximum value. In some embodiments, starting at a reference time (e.g., at the beginning of recording the sensor values, when there is a motor speed (e.g., P-level) change, following calibration of a reference value for the sensor signal), the minimum signature value and the maximum signature value may be set to the same value (e.g., a reference value). In subsequent iterations of the determining the signature in act 312, the minimum or maximum signature values may be updated in act 314 according to various conditions as described herein.

Process 300 then proceeds to act 316, where it may be determined whether a range, determined as the difference between the minimum signature value and the maximum signature value, is greater than a threshold value. In some embodiments, the threshold value may be the same for all motor speeds (e.g., P-levels). In other embodiments, a different threshold value may be used for different motor speeds. In some embodiments, the threshold value may change over time (e.g., the threshold value may increase over time to allow for more sensor drift over time). In some embodiments, the threshold value may be reset at a reference time (e.g., at the beginning of recording the sensor values, when there is a motor speed (e.g., P-level) change, following calibration of a reference value for the sensor signal), when the minimum signature value and the maximum signature values are also reset.

If it is determined in act 316 that the range is not greater than the threshold value, process 300 may return to act 312, where a signature is determined for a new observation time window. Acts 312, 314 and 316 may then repeated until it is determined in act 316 that the range for the signature is greater than the threshold value or when a reset/recalibration event occurs (e.g., a motor speed change). If it is determined in act 316 that the range is greater than the threshold value, sensor drift may be detected, and process 300 may proceed to act 318, where an action is performed to address the sensor drift. In some embodiments, performing an action includes outputting an indication that the sensor drift is out of range and should be corrected (e.g., by a manual recalibration or reset of the reference value). For instance, an alert may be displayed on a user interface associated with the controller of the cardiac support system to inform the user that a recalibration of the reference value for the sensor signal should be performed. As another example, the controller may be configured to initiate a recalibration of the reference value for the sensor signal to correct the sensor drift. In yet another example, both recalibration (e.g., automatic recalibration) and outputting an alert that the recalibration has been performed may be performed in act 318.

FIG. 4 illustrates a process 400 for a first technique for detecting sensor drift in a sensor signal (e.g., a differential pressure signal), in accordance with some embodiments of the present disclosure. Process 400 may begin in act 410, where a sensor signal (e.g., a differential pressure signal) is received (e.g., by a controller) from a heart pump. Process 400 may then proceed to act 412, where a sum S(t) is calculated according to the following formula:

$$S ( t ) = {\sum }_{i = 0}^{n} dP ( t - i ) ,$$

where n is the number of samples in the observation time window and dP is the sensor signal (in this case a differential pressure sensor signal). In some embodiments, n may be equal to 255 such that 256 samples present in the observation time window are considered in the summation at time 1. At a sampling rate of 25 Hz, such a 256 sample observation time window corresponds to a summation over approximately 10 seconds. Calculating the sum S(t) may be considered similar to applying a low-pass filter to the sensor signal to smooth out high frequency components of the signal. By smoothing out the high frequency components, the signature may be used to approximate the low frequency behavior of the signal.

Process 400 may then proceed to act 414, where a maximum value for the sum (Sₘᵤₓ) and a minimum value for the sum (Sₘᵢₙ) may be updated based on the sum S(t) calculated in act 412. As described in connection with act 314 of process 300, a minimum signature value (Sₘᵢₙ in this case) and a maximum signature value (Sₘₐₓ in this case) may be stored and updated when a new signature value is determined if the newly-determined signature value is less than the stored minimum value or is greater than the stored maximum value. Additionally, starting at a reference time (e.g., at the beginning of recording the sensor values, when there is a motor speed (e.g., P-level) change, following calibration of a reference value for the sensor signal), the minimum signature value (e.g., Sₘᵢₙ) and the maximum signature value (e.g., Sₘₐₓ) may be initialized to the same value. For instance, stored values may be set as S_{ref}=Sₘₐₓ=Sₘᵢₙ=S(t_{cal}). In subsequent iterations of determining S(t) in act 412, the minimum or maximum signature values may be updated in act 414 as follows:
If S(t)>Sₘₐₓ, Sₘₐₓ is set to S(t). Else, Sₘₐₓ is unchanged
If S(t)<Sₘᵢₙ, Sₘᵢₙ is set to S(t). Else, Sₘᵢₙ is unchanged

In this way, updating Sₘᵢₙ Or Sₘₐₓ in act 414 enables tracking the upper and lower bounds of S(t) over time as new values of S(t) are determined in act 412.

Process 400 may then proceed to act 416, where it may be determined whether the range Sₘₐₓ-Sₘᵢₙ is greater than a threshold value T_{drift}. As described in connection with process 300 shown in FIG. 3, the threshold value (e.g., T_{drift}) may be the same for all motor speeds, or a different threshold value may be used for different motor speeds. In some embodiments, the threshold value may change over time (e.g., the threshold value may increase over time to allow for more sensor drift over time). In some embodiments, the threshold value may be reset at a reference time (e.g., at the beginning of recording the sensor values, when there is a motor speed (e.g., P-level) change, following calibration of a reference value for the sensor signal), when the minimum signature value and the maximum signature values may also be reset. If it is determined in act 416 that the range Sₘₐₓ-Sₘᵢₙ is not greater than the threshold value T_{drift}, process 400 may return to act 412, where a value of S(t) may be calculated for a new observation time window. Acts 412, 414 and 416 may then repeated until it is determined in act 416 that the range is greater than the threshold value or when a reset/recalibration event occurs (e.g., a motor speed change). If it is determined in act 416 that the range is greater than the threshold value, sensor drift may be detected, and process 400 may proceed to act 418, where an action may be performed to address the sensor drift, non-limiting examples of which are described herein. In the example process 400, an indication of the detected sensor drift may be output as the action that is performed.

FIG. 5 illustrates a process 500 for a second technique for detecting sensor drift in a sensor signal (e.g., a differential pressure signal), in accordance with some embodiments of the present disclosure. In some embodiments, a cardiac support system may be configured to store data associated with one or more sensor signals (e.g., real-time sensor data). In some embodiments, the cardiac support system may additionally or alternatively be configured to calculate and store derived data based on the sensed (e.g., real-time) sensor data. For instance, the system may be configured to calculate minimum, maximum, and/or mean values for a differential pressure sensor signal within a particular time window (e.g., 1 minute), and the calculated values for the statistical measure may be recorded in log that is stored by the system. In such an instance, the mean value of the differential pressure signal may be considered as values MeanPlacement(t) in a series of mean placement signals. In some embodiments, the mean placement signals may be used as a signature to detect drift in the differential pressure signal.

Process 500 may begin in act 510, where the mean placement signal MeanPlacement(t) is received (e.g., by a controller accessing a log with the stored mean placement signal). Process 500 may then proceed to act 514, where a maximum value for the mean placement signal (MPₘₐₓ) and a minimum value for the sum (MPₘᵢₙ) may be updated based on the received mean placement signal MeanPlacement(t). As described in connection with act 314 of process 300, a minimum signature value (MPₘᵢₙ in this case) and a maximum signature value (MPₘₐₓ in this case) may be stored and updated when a new signature value (e.g., MeanPlacement(t) is determined if the newly-determined signature value is less than the stored minimum value or is greater than the stored maximum value. Additionally, starting at a reference time (e.g., at the beginning of recording the sensor values, when there is a motor speed (e.g., P-level) change, following calibration of a reference value for the sensor signal), the minimum signature value (e.g., MPₘᵢₙ) and the maximum signature value (e.g., MPₘₐₓ) may be initialized to the same value. For instance, stored values may be set as MP_{ref}=MPₘₐₓ=MPₘᵢₙ=MeanPlacement(t_{cal}). In subsequent iterations, the minimum or maximum signature values may be updated in act 512 as follows:
If MeanPlacement(t)>MPₘₐₓ, MPₘₐₓ is set to MeanPlacement(t). Else, MPₘₐₓ is unchanged.
If MeanPlacement(t)<MPₘᵢₙ, MPₘᵢₙ is set to MeanPlacement(t). Else, MPₘᵢₙ is unchanged

In this way, updating MPₘᵢₙ or MPₘₐₓ in act 512 enables tracking the upper and lower bounds of MeanPlacement(t) over time as new values of MeanPlacement(t) are received in act 510.

Process 500 may then proceed to act 514, where it may be determined whether the range MPₘₐₓ-MPₘᵢₙ is greater than a threshold value TMP. As described in connection with process 300 shown in FIG. 3, the threshold value (e.g., TMP) may be the same for all motor speeds, or a different threshold value may be used for different motor speeds. In some embodiments, the threshold value may change over time (e.g., the threshold value may increase over time to allow for more sensor drift over time). In some embodiments, the threshold value may be reset at a reference time (e.g., at the beginning of recording the sensor values, when there is a motor speed (e.g., P-level) change, following calibration of a reference value for the sensor signal), when the minimum signature value and the maximum signature values may also be reset. If it is determined in act 514 that the range MPₘₐₓ-MPₘᵢₙ is not greater than the threshold value TMP, process 500 may return to act 510, where a value of MeanPlacement(t) may be received for a new observation time window. Acts 510, 512 and 514 may then repeated until it is determined in act 514 that the range is greater than the threshold value or when a reset/recalibration event occurs (e.g., a motor speed change). If it is determined in act 514 that the range is greater than the threshold value, sensor drift may be detected, and process 500 may proceed to act 516, where an action may be performed to address the sensor drift, non-limiting examples of which are described herein. In the example process 500, an indication of the detected sensor drift may be output as the action that is performed.

FIG. 6A illustrates a plot of an example mean placement signal for a differential pressure (dP) sensor over a time period of 2 days. FIG. 6B illustrates a plot of the mean motor current over the same time period as the plot shown in FIG. 6A. As shown in FIG. 6A, the mean placement signal drifts considerably over the 2 day time period, whereas the mean motor current shown in FIG. 6B remains relatively constant at a constant motor speed.

The plot in FIG. 6A shows as a function of time, the mean placement signal (MeanPlacement(t)), the maximum signature value (MPₘₐₓ), and the minimum signature value (MPₘᵢₙ) determined in accordance with process 500 shown in FIG. 5. Also shown in FIG. 6A is a threshold value (TMP) 610. In the example shown in FIG. 6A, the threshold value is constant across both motor speeds illustrated in the figure. In some embodiments, the threshold value may be changed when the motor speed changes and/or over time, as described herein. FIG. 6A also shows the determined range (MPₘₐₓ-MPₘᵢₙ) 612 of the signature during each of a plurality of observation time windows, wherein the height of vertical bars in the figure represents a magnitude of the range. As described with reference to act 514 of process 500, when the magnitude of the range exceeds the threshold value, sensor drift is detected.

FIG. 6A illustrates that sensor drift is detected earlier using the techniques described herein relative to the conventional approach of a user noticing a discrepancy and initiating a manual calibration of a reference value for the pressure signal. For instance, as shown in FIG. 6A, sensor drift is detected at time 618, which is before the time 620 when a user-initiated manual calibration was performed. As another example, sensor drift was detected at time 628, which is before the time 630 when a user-initiated manual calibration was performed. By detecting sensor drift earlier than is typically achieved using conventional methods, some embodiments of the present disclosure may take action to address the sensor drift in a more timely manner (e.g., by performing an autocalibration routine and/or informing the user about the sensor drift), which may result in the cardiac support device providing more accurate information to a user of the cardiac support device, thereby improving patient care.

In some embodiments, when sensor drift is detected using one or more of the sensor drift detection techniques or in response to receiving input via a user interface that a user has detected sensor drift, the sensor drift may be corrected (also referred to herein as “recalibration of a reference value for a sensor signal”). Some conventional techniques for correcting for sensor drift associated with pressure signals of a heart pump device include significantly reducing the pump speed (e.g., to P-1 or P-2) and waiting a predetermined amount of time to let the pressure signal “settle.” The inventors have recognized that such an approach may not be advisable in all scenarios, particularly if reducing the pump speed will not provide adequate support for the patient. To this end, in some embodiments, sensor drift correction may be performed without reducing the pump speed or requiring only small reductions in pump speed. In some embodiments, the sensor drift may be corrected using an automated process. In other embodiments, an alert may be generated and displayed to the user to inform the user to initiate manual correction process.

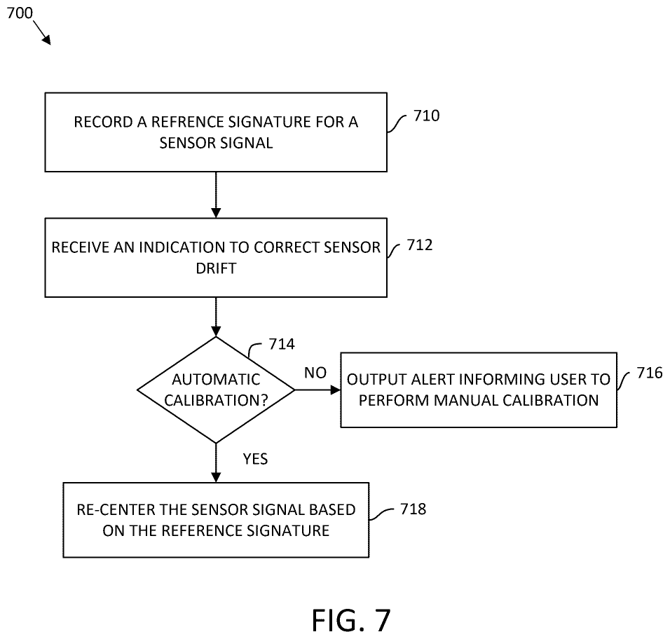

FIG. 7 illustrates a process 700 for correcting sensor drift associated with a pressure sensor (e.g., a differential pressure sensor) of a heart pump, in accordance with some embodiments of the present disclosure. Process 700 may begin in act 710, where a reference signature for a sensor signal (e.g., a differential pressure sensor signal) is recorded. For instance, the reference signature may be recorded at a reference time in response to an occurrence of a reference event (e.g., at the beginning of recording the sensor values, when there is a motor speed (e.g., P-level) change, following calibration of a reference value for the sensor signal). The vertical lines in the plots of FIGS. 6A and 6B represent times at which reference events occurred while a real-time differential pressure signal was processed in accordance with the techniques described herein. Each time a reference event occurs, a new reference signature may be recorded and used for sensor drift correction when sensor drift is detected. In some embodiments, the reference signature may be computed as a sum (S_{ref}) of pressure signal values over a time window of pressure sensor samples (e.g., 256 samples). In other embodiments, the reference signature may be determined based on a mean placement signal (MP_{ref}), which represents a mean of differential pressure signals over a time window (e.g., one minute). As described herein, a controller associated with a heart pump may be configured to store real-time sensor data and/or derived (e.g., mean, minimum, maximum) sensor data. At least some of such stored data may be used to determine the reference signature for the sensor signal in act 710 rather than having to calculate the reference signature when a reference event occurs.

Process 700 may then proceed to act 712, where an indication to correct sensor drift is received. For instance, when one of the data-driven techniques for detecting sensor drift described herein (e.g., the range of a tracked signature exceeds a threshold value) are used to detect the sensor drift, the controller used to detect the sensor drift may provide the indication to correct sensor drift. In other embodiments, in which data-driven techniques for detecting sensor drift are not user, a user may provide the indication to correct sensor drift, for example, via a user interface associated with the controller of the heart pump system. As should be appreciated, the indication to correct the sensor data may be received at a second time after a first time when the reference signature for the sensor signal is recorded. When the indication is received in act 712, the reference signature most recently recorded for the sensor signal may be used to perform the sensor drift correction.

In response to receiving the indication in act 712 and therefore determining that sensor drift correction is needed, process 700 may then proceed to act 714, where may be determined whether to perform automatic calibration of the reference value associated with the sensor signal. The determination of whether to perform automatic calibration may be made based on any suitable factors. Because the automatic calibration routine may not require a modification to the pump speed (and therefore may have little or no impact on patient support), some heart pump systems may be configured to always perform automatic calibration unless certain conditions are met. For instance, the controller may be configured to determine a number of sensor drift corrections that happen within a particular amount of time, and if that number exceeds a particular threshold value, it may be determined that a manual calibration should be used (e.g., a manual calibration that reduces the pump speed to let the sensor signal settle). Some heart pump systems may be configured to disable automatic calibration to enable the user of the system to have more control over the decision of when to perform the sensor drift correction. For such systems, it may always be determined in act 714 not to perform automatic calibration unless one or more conditions are met (e.g., automatic calibration is activated on the system).

If it is determined in act 714 not to perform automatic calibration, process 700 may proceed to act 716, where an alert may be output to the user to make the user aware that the sensor drift should be corrected using a manual calibration process. In some embodiments, the manual calibration process may be performed similarly to convention sensor drift correction techniques (e.g., reducing the pump speed to allow the sensor signal to settle). In other embodiments, the manual calibration process may be performed similarly to how automatic calibration is performed, which is described in more detail below. However, rather than being performed automatically in response to detecting that sensor drift correction is needed, the manual calibration process may need to be initiated by a user interacting with a user interface associated with the heart pump system. For instance, an alert may be presented on the user interface and the alert may provide the user the option to perform the calibration by selecting a user interface element displayed on the user interface. The user may then interact with the user interface element to initiate the drift sensor correction.

If it is determined in act 714 that automatic calibration is to be performed, process 700 may proceed to act 718, where a sensor drift correction process is initiated. For instance, sensor drift correction may be performed based on the most recently recorded reference signature (e.g., S_{ref} or MP_{ref}) by re-centering a real-time sensor signal (e.g., dP(t)) based on the reference signature according to one of the following equations based on whether the summation technique or mean placement technique is used:
dP′(t)=dP(t)−(S(t)−S_{ref})/N, where N is the number of samples used for the sum; or
dP′(t)=dP(t)−(MeanPlacement(t)−MP_{ref}).

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of correcting sensor drift associated with a pressure sensor of a heart pump, the method comprising:
recording, at a first time, a first reference signature for a pressure signal received from the pressure sensor;
receiving, at a second time, an indication to correct sensor drift associated with the pressure sensor, wherein the second time is after the first time;
receiving a real-time pressure signal from the pressure sensor;
adjusting, by a controller associated with the heart pump, the real-time pressure signal based on the first reference signature in response to receiving the indication to correct sensor drift; and
displaying the adjusted real-time pressure signal.

2. The method of claim 1, wherein the pressure sensor comprises a differential pressure sensor.

3. The method of claim 2, wherein the heart pump is inserted across a pulmonary valve of a heart of a patient, and the pressure signal is a differential pressure signal across the pulmonary valve.

4. The method of claim 1, wherein recording the first reference signature comprises calculating a sum of pressure signal values within a time window of the pressure signal, the time window including the first time.

5. The method of claim 1, wherein recording the first reference signature comprises determining a mean placement signal within a time window of the pressure signal, the time window including the first time.

6. The method of claim 1, wherein receiving the indication to correct sensor drift in the pressure signal comprises receiving user input via a user interface associated with the heart pump.

7. The method of claim 1, further comprising:
detecting, based on an analysis of the pressure signal by the controller, the sensor drift,
wherein receiving the indication to correct sensor drift in the pressure signal comprises receiving the indication from the controller in response to detecting the sensor drift.

8. The method of claim 1, wherein automatically adjusting the real-time pressure signal based on the first reference signature in response to receiving the indication to correct sensor drift comprises:
re-centering the real-time pressure signal based on the first reference signature.

9. The method of claim 8, wherein re-centering the real-time pressure signal based on the first reference signature comprises subtracting or adding an offset value based on the first reference signature to the real-time pressure signal.

10. The method of claim 1, further comprising:
determining whether to perform automatic drift calibration; and
outputting an alert on a user interface associated with the heart pump when it is determined not to perform automatic drift calibration.

11. The method of claim 1, wherein adjusting the real-time pressure signal based on the first reference signature is performed without adjusting a speed of the heart pump.

12. The method of claim 1, further comprising:
recording, at the second time, a second reference signature for the pressure signal;
receiving, at a third time, an indication to correct sensor drift associated with the pressure sensor, wherein the third time is after the second time; and
adjusting, by the controller associated with the heart pump, the real-time pressure signal based on the second reference signature in response to receiving, at the third time, the indication to correct sensor drift.

13. The method of claim 1, further comprising:
detecting, at a third time, a change in a speed of the heart pump, wherein the third time is after the first time;
recording, at the third time, a second reference signature for the pressure signal;
receiving, at a fourth time, an indication to correct sensor drift associated with the pressure sensor, wherein the fourth time is after the third time; and
adjusting, by the controller associated with the heart pump, the real-time pressure signal based on the second reference signature in response to receiving, at the fourth time, the indication to correct sensor drift.

14. The method of claim 1, further comprising:
determining at a third time, between the first time and the second time, a signature within a time window of the pressure signal;
updating a minimum signature value or a maximum signature value based, at least in part, on the signature within the time window; and
determining whether a range of the signature is greater than a threshold value, wherein the range is determined as a difference between the minimum signature value and the maximum signature value,
wherein an indication to correct sensor drift associated with the pressure sensor comprises receiving the indication in response to determining that the range of the signature is greater than the threshold value.

15. The method of claim 14, wherein determining a signature within a time window of the pressure signal comprises:
determining a sum of pressure signal values within the time window of the pressure signal.

16. The method of claim 14, wherein determining a signature within a time window of the pressure signal comprises:
determining a mean value of pressure signal values within the time window of the pressure signal.

17. The method of claim 16, further comprising:
initializing, at the first time, the minimum signature value and the maximum signature value to a same value,
wherein updating the minimum signature value and/or the maximum signature value based, at least in part, on the signature comprises:
updating the minimum signature value to the mean value when the mean value is less than the minimum signature value, or
updating the maximum signature value to the mean value when the mean value is greater than the maximum signature value.

18. The method of claim 16, wherein a length of the time window is one minute.

19. A heart pump system, comprising:
a heart pump including a pressure sensor configured to sense a pressure within a portion of a heart of a patient; and
a controller configured to:
record, at a first time, a first reference signature for a pressure signal received from the pressure sensor;
receive, at a second time, an indication to correct sensor drift associated with the pressure sensor, wherein the second time is after the first time;
receive a real-time pressure signal from the pressure sensor;
adjust, by a controller associated with the heart pump, the real-time pressure signal based on the first reference signature in response to receiving the indication to correct sensor drift; and
display the adjusted real-time pressure signal.

20-36. (canceled)

37. A controller for a heart pump system, the controller comprising:
at least one hardware processor configured to:
record, at a first time, a first reference signature for a pressure signal received from a pressure sensor associated with the heart pump system;
receive, at a second time, an indication to correct sensor drift associated with the pressure sensor, wherein the second time is after the first time;
receive a real-time pressure signal from the pressure sensor;
adjust the real-time pressure signal based on the first reference signature in response to receiving the indication to correct sensor drift; and
display the adjusted real-time pressure signal.

38-54. (canceled)
